(19) United States
(12) Patent Application Publication
(10) Pub. No.: US 2019/0080242 A1
(43) Pub. Date: Mar. 14, 2019

(54) Method that interconnects user or machines by minds profile.

(71) Applicant: Flávio Antônio da Costa Filho, São Paulo (BR)

(72) Inventor: Flávio Antônio da Costa Filho, São Paulo (BR)

(21) Appl. No.: 15/926,611

(22) Filed: Mar. 20, 2018

Related U.S. Application Data
(60) Provisional application No. 62/477,198, filed on Mar. 27, 2017.

(51) Int. Cl. G06N 3/08; G06F 15/18; A61B 5/16

(52) U.S. Cl. CPC G06N 3/086; A61B 5/165; G06F 15/18

(57) ABSTRACT

A method that interconnects user or machines by minds profile is disclosed. This method uses an algorithm, a programming language, a database and a digital or electronic device to create a mental profile of individuals or characters. A series of logical rules are then applied to allow this mental profile to learn content, evolve, and relate to other profiles. This gives a new depth to the digital market by relating people and content through mental profiles. It can function as an artificial intelligence for machines.

From provisional application “Programming algorithm that interconnects user minds”, application Ser. No. 62/477,198, confirmation number 9296.

### BACKGROUND OF THE INVENTION

The success of the internet worldwide has proliferated technologies that can help in the process of searching for information. This information returns in text format or another type of digital document. In addition, distributing relevant content on the internet takes into consideration the user's browsing history, searches, accessed websites, often invading their privacy.

The process of searching for information and distributing relevant content to network users raises many challenges, such as knowing the authenticity of the browsing history and user interests and finding the most relevant document to bring the requested information. These challenges open space for new technologies to be developed capable of bringing more efficient responses to the search processes, as well as distributing relevant content with more assertiveness.

One of these new technologies is this method able to bring as a result of a search for information, not documents but a user who presents an efficient mental profile to relate to the user that originated the demand besides having knowledge related to this demand. This method is also capable of distributing content based on this mental profile and not just browsing history on the internet.

### BRIEF SUMMARY

In short, this method uses a algorithm, programming language, a database and digital or electronic devices to generates a “mental digital identity” of the user that expresses his behavioral psychological profile and the knowledge generated and learned. This digital mental identity, here called “MIND ID”, can be used for a wide range of purposes related to associating people, associating information, behavioral prediction, solving problems in a wide variety of areas, statistics, research, content distribution, market, commercial and marketing planning among other purposes.

The “MIND ID” is composed of two parts: psychological cognitive behavioral expression and content.

The first step is to create a “MIND ID” by applying a psychological test or behavioral analysis of the user in an application that uses this algorithm. This “MIND ID” is the result of a series of logical evaluations of applied testing or user behavior, which generate variables. These variables are given a value from 0 to 100, per example. The set of these variables defines a “MIND ID”.

The second step is to feed the “MIND ID” with content through scripts associated with softwares, hardware or any other devices that processes the information generated by the user and feeds a database. This processing takes the information, whether visual or textual or any other type that can be digitally processed, applies a sequence of logical filters to extract the idea behind the content. Associate these extracted ideas with the user's “MIND ID”.

The third step is to feed back the “MIND ID” transforming it by changing the values of the variables that compose it according to the behavior and information exchanged over time by the user.

The fourth step is to relate “MIND ID”. For this to happen a series of logical rules detects other “MIND ID” with characteristics that favor the attendance of demands generated by this “MIND ID” in any type of device or interface.

These steps may or may not occur sequentially.

Therefore, this method can be used as an artificial intelligence capable of generating mental profiles, relating them, evolving them and learning from them. It functions as a logical library that can be associated with devices, softwares or hardwares for the most diverse market applications.

### DETAILED DESCRIPTION

The invention will be detailed based on the steps already mentioned: creation of “MIND ID”, generation of content for “MIND ID”, evolution of “MIND ID”, relations between “MIND ID” and content distribution.

For this method to be applied and developed it is necessary an algorithm, programming language, database and a digital or electronic device.

Creating “MIND ID”: As mentioned, a “MIND ID” is a set of variables that represent an individual, user or character. This representation is psychological cognitive behavioral also based on studies of neuro science. Each of these variables represents a psychological aspect of the user and can have any value determined by the algorithm based on the response of a psychological test or in reading the behavior of the user within the system that the algorithm is applied to. This set of variables with any name, for example: X, Y, Z, K, E, P, Q are associated with registering this user in a database.

Generating content for “MIND ID”: With the “MIND ID” created a series of secondary associations begin to happen within a database. Through a script applied to websites, mobile applications, softwares or hardware the algorithm processes the information exchanged by this “MIND ID” and associates it with its registration in the database. For example, the user uses a chat to talk to a friend, in this chat there is the inclusion of the algorithm script, the script executes the algorithm and collects the information exchanged in this dialog. With the information collected are broken into pieces and the algorithm recognizes the grammatical and semantic function of words, or records photos and videos and associates all these pieces giving a numerical value for each of these associations. This is recorded in the database and associated with the “MIND ID”.

Evolution of “MIND ID”: Any interaction of “MIND ID” within systems where the algorithm is used generates information that is processed by the script and associated with the “MIND ID”. This information can change the value initially given to the variables that make up the “MIND ID”, evolving the information over time. In addition to constantly registering more associations between content, or generated information, and “MIND ID”. These changes update the database where the “MIND ID” is registered.

Relationships between “MIND ID” and content distribution: a set of rules based on neuro-science studies are converted into logical rules capable of determining efficiency in the relationship between “MIND ID” for certain purposes besides detecting the type of content relevant to each “MIND ID”. For example imagine a social network in the internet where the script of the algorithm is applied. A user of this network with his/her “MIND ID” searches for information on how to solve an accounting problem in the company. Through the rules of relations and distribution of content the algorithm will locate other “MIND ID” with or without knowledge of the subject and that has an efficient mental profile to help the user who generated the problem to resolve the issue. Another example is a user who subscribes to a news portal on the internet. He receives news on his cell phone that relates to his “MIND ID” and not his browsing history on the internet. This is because all information exchanged and recorded in the database is associated with “MIND ID”. Statistical calculations are applied to ensure that new news reaches users who have a relationship with the subject matter based on the “MIND ID” content history of all users with the same or related information.

It should be noted that aspects of the invention may be incorporated as a computer program system, method or product, but not limited thereto. As a logical model can be applied on any device that allows the execution of logical commands. Accordingly, aspects of the invention may take the form of a hardware entirely embodiment, a software entirely embodiment (including firmware, resident software, microcode, etc.) or an embodiment combining aspects of software and hardware that may be generally referred to here as a “circuit”, “module” or “system”. Or it can take the form of a smartphone app, or smarttv application, or smart device app.

To make clear, what is being object of patent is a method of relating people and information based on the creation of a digital mental identity with its own characteristics and learning ability. This identity and the rules of relation between them can be applied for various purposes. Such as a PHP script for an internet software or a new marketing research platform for example. Or it can be written in another programming language like C and applied in conjunction with electronic circuits capable of running software on toys or other devices.

The big question is that this invention gives a digital mental identity to individuals or characters, and creates digital processes that mimic the human cognitive process and allows the relationship between these identities and the content they generate. And this method applied in a computer algorithm can also teach a machine and generate for it a mental identity, with artificial intelligence.

This disclosure has been provided for purposes of exemplification and description but is not intended to be exhaustive or limiting. Many modifications and variations will be apparent to those skilled in the art. The embodiments have been chosen and described to explain principles and practical application, and to enable other persons skilled in the art to comprise the disclosure of various embodiments with various modifications which are suitable for the particular use contemplated.

Although the illustrative embodiments of the invention have been described herein with reference to the given examples, it is to be understood that the embodiments of the invention are not limited to such precise embodiments, and that various other changes and modifications may be effected by a skilled person in art without departing from the reach or spirit of the disclosure.

What is claimed is:

1. A method of creating a digital mental profile that allows people and content to be related. This method uses an artificial intelligence algorithm that through a programming language, a database and a digital or electronic device creates a mental digital identity through the processing of information received from a psychological test or analysis of user behavior. This digital mental profile is able to learn information and evolve. The method allows the relationship between these profiles to efficiently meet specific demands for information by placing compatible profiles for a certain function in contact. This digital mental profile can be used for various purposes including marketing. This method is capable of teaching machines with artificial intelligence.
